(19) United States
(12) Patent Application Publication
(10) Pub. No.: US 2014/0243726 A1
(43) Pub. Date: Aug. 28, 2014

(54) HEMOSTATIC AND BIODEGRADABLE BANDAGES AND BANDAGE IMPLANTS

(71) Applicant: ORTHOVITA, INC., Malvern, PA (US)

(72) Inventors: James San Antonio, Media, PA (US); Robert J. Chiasera, JR., Downington, PA (US)

(73) Assignee: ORTHOVITA, INC., Malvern, PA (US)

(21) Appl. No.: 13/778,993

(22) Filed: Feb. 27, 2013

(51) Int. Cl. A61L 15/32

(52) U.S. Cl. CPC A61L 15/325
USPC 602/50

(57) ABSTRACT

In one embodiment, the present invention is a multi-layered bandage including a wound-contacting first layer which is porous, fluid-permeable, biodegradable, and capable of accelerating hemostasis and wound healing, and a second layer formed from an absorbent material which is releasably secured to the first layer. The first layer includes a porous, fluid-permeable, biodegradable lattice structure formed from a homogeneous mixture of collagen and a clotting enzyme which, as healing occurs, may be replaced by new tissue. The second layer may be detached at a time deemed safe, usually immediately after hemostasis occurs, without disturbing the healing process.

### BACKGROUND OF THE INVENTION

The management and treatment of wounds, including those resulting from surgical procedures, is an area of great interest in the medical industry and has been the subject of significant research and innovation. Current technologies pertaining to bandages are replete with innovations relating to protection, absorbency, delivery, removal, adherence, medication, and maintenance of the wound environment. It is clear from past innovations in the field that the design of an effective bandage depends on many factors, including for example, the facilitation and acceleration of healing, maintenance of an environment conducive to healing, prevention of bacterial infection, protection against reinjury, and prevention of reinjury upon removal.

Although bandages as are known in the art provide for some of the above factors, the need for an improved bandage which can meet all of the above criteria still exists.

### BRIEF SUMMARY OF THE INVENTION

The present invention relates to wound dressings generally, and more particularly to multi-layered bandages and bandage implants that include a hemostatic bioabsorbable portion and a detachable non-resorbable portion. The present invention bandages are aesthetic and integrate with the wound upon contact to facilitate healing. Once applied, only the biodegradable portion of the bandage remains on the wound/at the wound site via interaction of a clotting enzyme that interacts with the wound. Unlike bandages in the art, synthetic adhesive is not used to adhere the bandage to the wound.

One embodiment of the present invention is a multi-layered bandage including a first wound-contacting layer made of a porous, biodegradable material containing a clotting enzyme, and a second absorbent layer releasably secured to the first layer, wherein the bandage allows flow of wound exudate into the first layer to interact with the clotting enzyme and, optionally, into the second layer for absorption. In a preferred embodiment, the first biodegradable layer comprises a collagen-thrombin lattice structure and the second layer is non-resorbable. The mixture of collagen and thrombin forming the lattice structure of the first layer may be homogeneous. Optionally, the first layer can include a plurality of channels extending from a bottom surface to a top surface with a plurality of flaps at the top surface adapted to cover the open ends of the channels at the top surface when manual pressure is applied to the bandage to press it against the wound.

In another embodiment, the present invention is a system for treating a wound including a biodegradable implant including a homogenous mixture of collagen and a clotting enzyme, the collagen forming a porous lattice structure and the clotting enzyme positioned throughout the lattice structure in a dry form, and an applicator releasably secured to the implant, the applicator including an absorptive matrix and an interface adapted to allow a user to apply compression through the applicator and against the implant. In a preferred embodiment, the implant contains collagen and thrombin. Optionally, the implant can include a plurality of channels extending from a bottom surface to a top surface with a plurality of flaps at the top surface adapted to cover the open ends of the channels at the top surface.

In a further embodiment, the present invention is a method for treating a wound including the steps of applying a bandage having a first biodegradable, wound-contacting layer and a second absorbent, non-resorbable layer, applying compression to the second layer to allow exudate to migrate from the wound into the first layer and, optionally, to the second layer, maintaining said compression until hemostasis occurs, and detaching the second layer from the first layer, thereby leaving the first layer in contact with the wound. In a preferred embodiment, the first layer contains a dry, homogenous mixture of collagen and a clotting enzyme, thereby allowing for hemostasis to occur at an interface between the first layer and the wound. In some instances, excess exudate may migrate from the wound, into the first layer, and through to the absorbent second layer, which can then absorb the exudate.

The first layer can include a plurality of channels extending from a bottom surface to a top surface with a plurality of flaps at the top surface adapted to cover the open ends of the channels at the top surface. If such channels and flaps are present, during the compression step, the exudate may migrate through the plurality of channels of the first layer. If the flaps are in a closed position, the exudate may be prevented from exiting through the top surface of the first layer. If the flaps are in an open position, the exudate may pass through the channels of the first layer, and out of the top surface of the first layer, and optionally into the second layer. In one instance, following the detachment of the second layer from the first layer, the flaps may be in a closed position. Upon removal, the second layer may be used to wipe excess exudate from and around the wound.

### BRIEF DESCRIPTION OF THE DRAWINGS

FIG. 1 is a cross-sectional view of one embodiment of the present invention.

FIG. 2a is a cross-sectional view of another embodiment of the present invention.

FIG. 2b is a cross-sectional view of an alternative embodiment of the present invention.

FIG. 2c is a cross-sectional view of yet another alternative embodiment of the present invention.

FIG. 2d is a cross-sectional view of a “drip” or “spot” process used to apply the clotting enzyme solution for the embodiments of FIGS. 2b and 2c.

FIG. 2e is a cross-sectional view of another alternative embodiment of the present invention.

FIG. 2f is a cross-sectional view of yet another alternative embodiment of the present invention.

FIG. 2g is a top perspective view of the first layer of the embodiment shown in FIG. 2f.

FIG. 2h is a cross-sectional view of yet another alternative embodiment of the present invention.

FIG. 2i is a cross-sectional view of another alternative embodiment of the present invention

FIG. 2j is a top-down view of the embodiment shown in FIG. 2i.

FIG. 3 is a side view of the embodiment shown in FIG. 1 applied to a wound.

FIG. 4 is a side view of the embodiment shown in FIG. 1 as a compression force is applied against the invention towards the wound.

FIG. 5 is a side view of the embodiment shown in FIG. 1 as the second layer is detaching from the first layer.

### DETAILED DESCRIPTION

As used herein, the term “wound” refers to any physiological trauma where soft body tissue is torn, cut, punctured, or otherwise broken, including but not limited to superficial trauma to skin, surgical incisions, trauma to internal organs and tissues, and the like. As used herein, the term “exudate” refers to blood, and related constituents, which is present in and seeps from a wound.

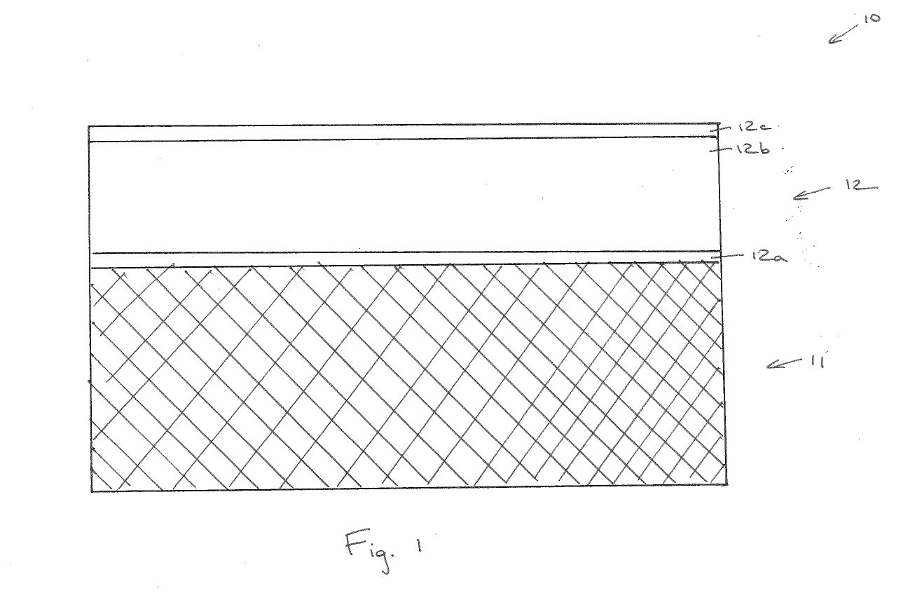

In one embodiment of the present invention as illustrated in FIG. 1, a multi-layered bandage 10 includes a first layer 11 and a second layer 12. The first layer 11 includes a porous, fluid-permeable, biodegradable lattice structure formed from a mixture of collagen and a clotting enzyme and, in certain embodiments, is skin-toned for aesthetic purposes. The second layer 12 is an absorbent layer releasably secured to the first layer 11. The second layer 12 includes an absorbent material 12b suitable for absorbing excess exudate which may migrate from the wound, into and through the first layer 11, and out of the first layer 11.

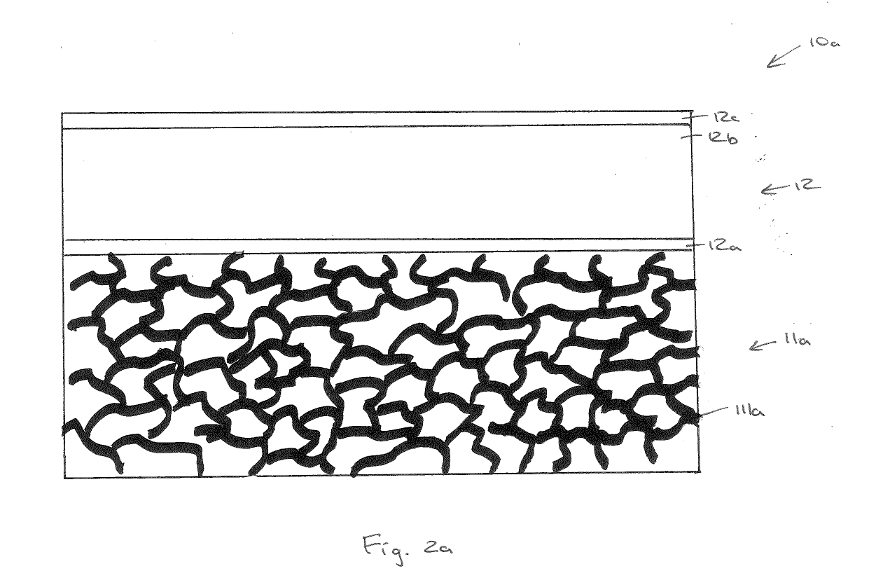

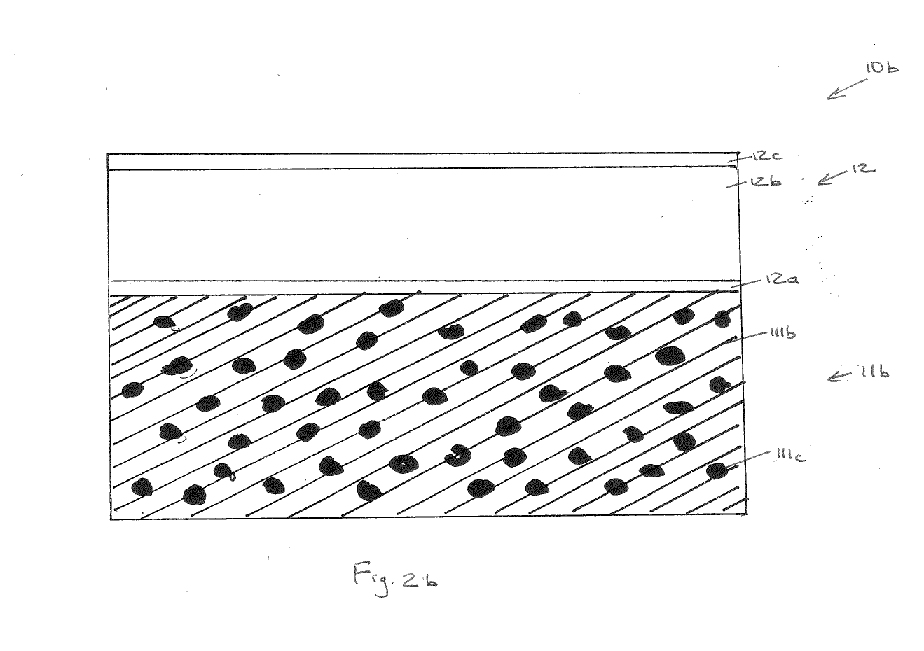

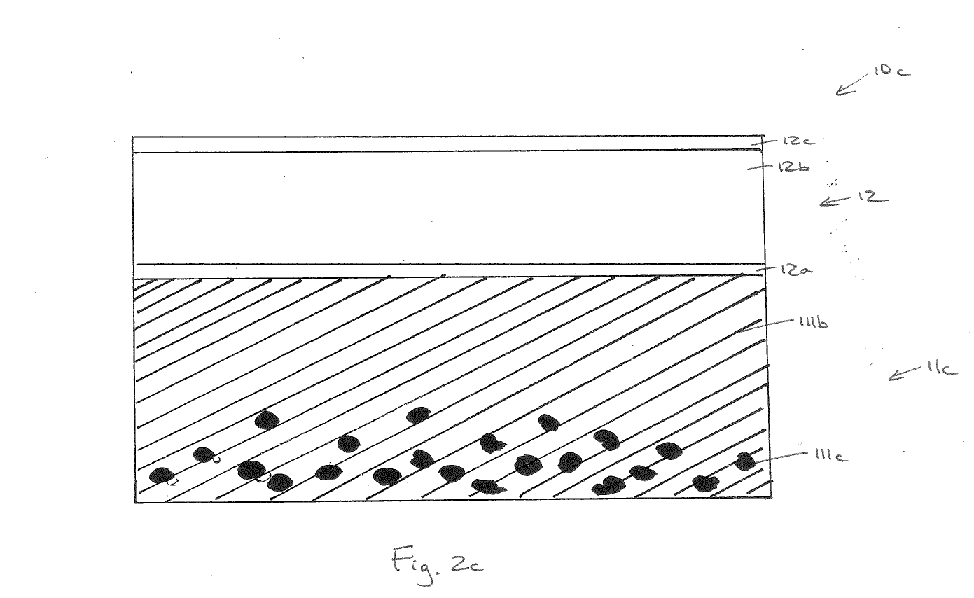

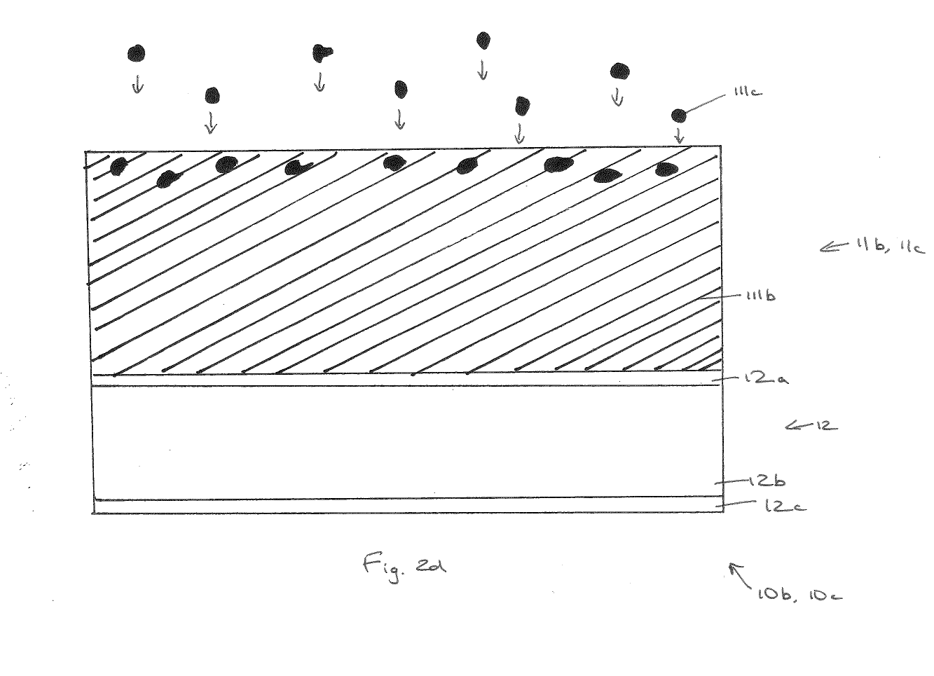

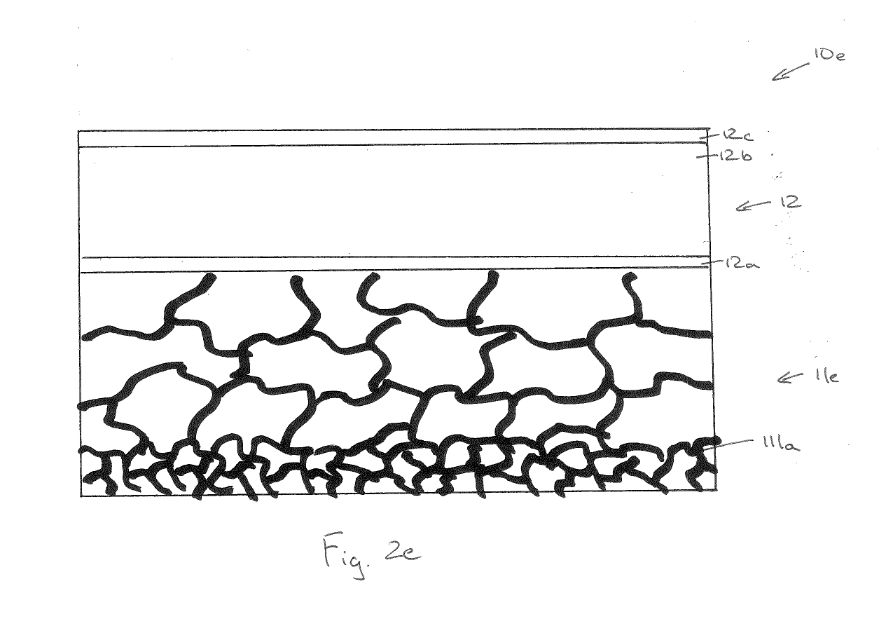

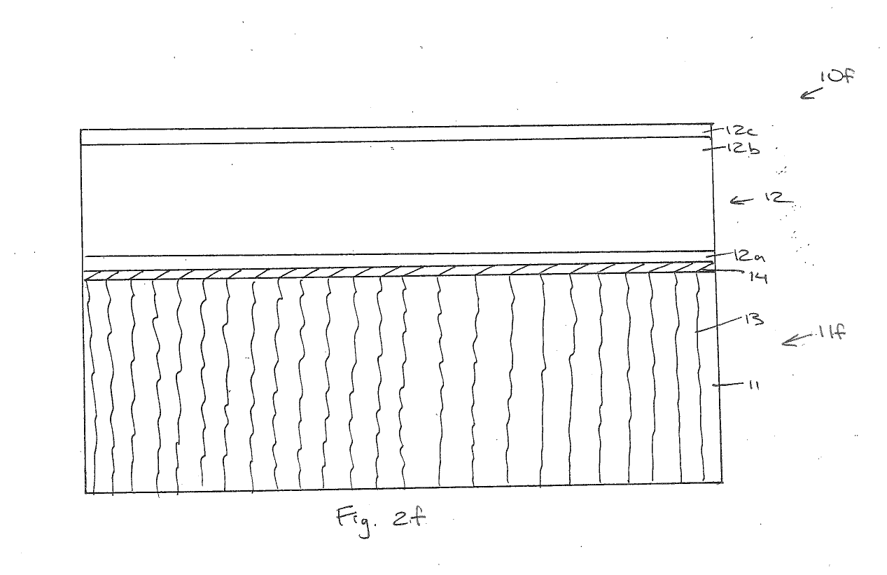

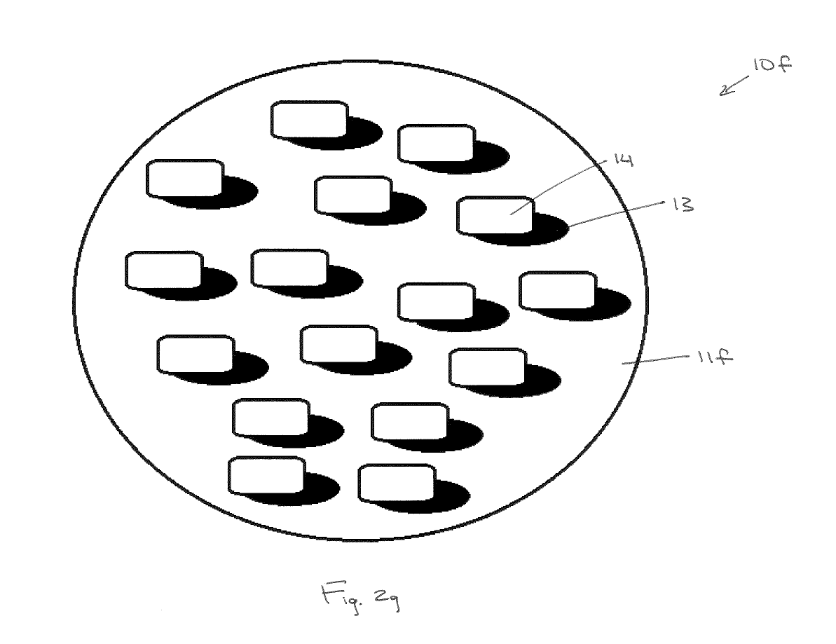

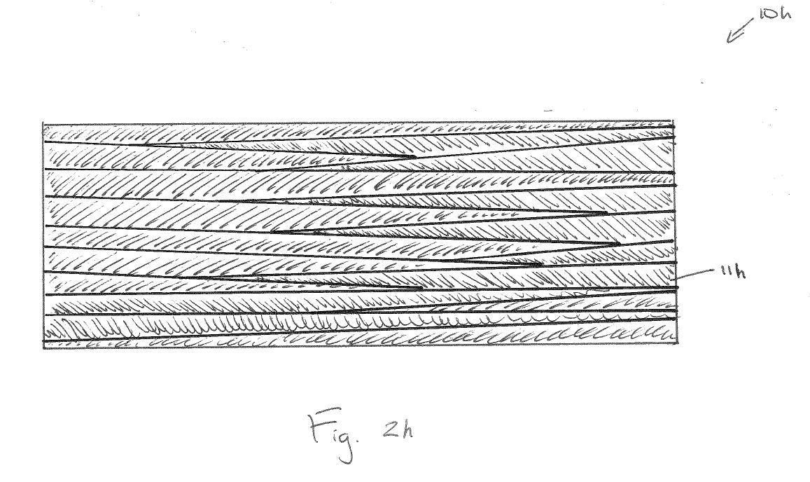

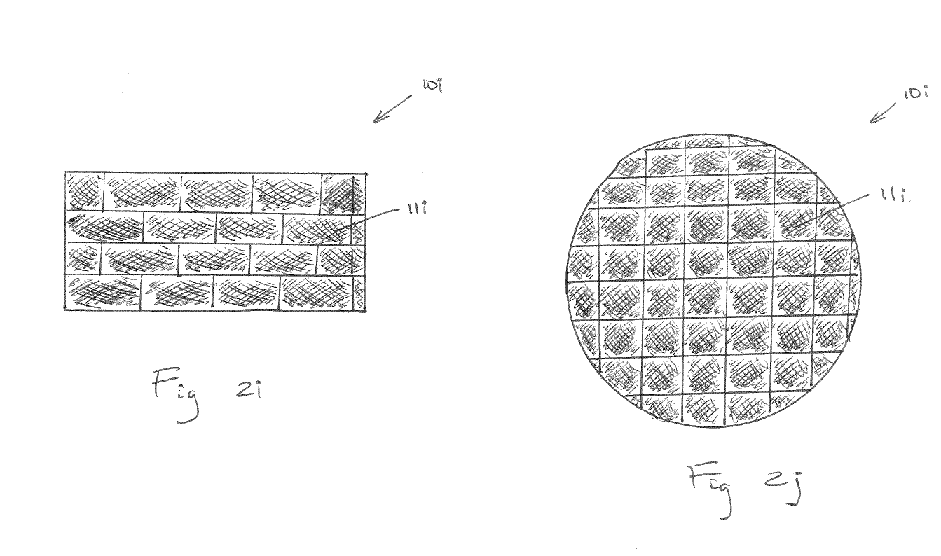

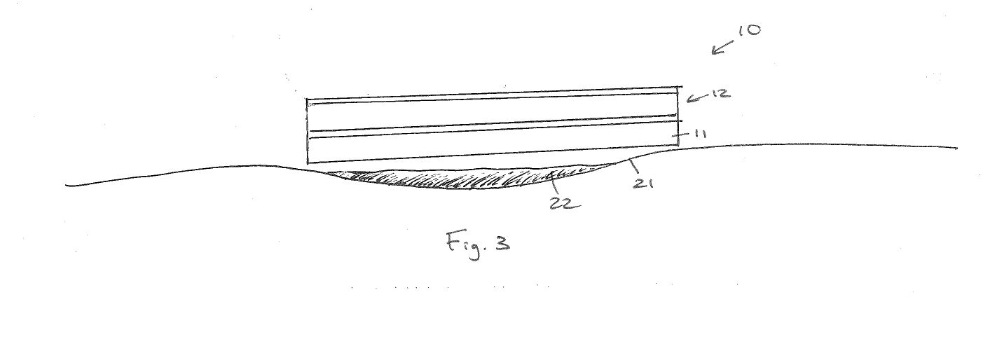

In a preferred embodiment, as illustrated in FIG. 2a, the lattice structure of the first layer 11a is formed from a homogenous mixture of collagen and a clotting enzyme 111a, preferably thrombin. Such a lattice structure may be formed by a variety of manufacturing methods. Furthermore, the first layer 11a may include additional materials such as polyethylene glycol or the like, which may also be homogeneously throughout the lattice structure. The lattice structure provides passageways throughout the first layer 11a for the interaction of the exudate from the wound with the clotting enzyme.

In one embodiment, as illustrated in FIG. 1, the second layer 12 can include a top surface 12c of, for example, an impermeable plastic sheeting, a central portion 12b of highly absorbent scaffolding material such as cotton or the like, and a bottom surface 12a of, for example, semi-permeable plastic that is non-resorbable and allows exudate to pass through and into the central absorbent portion. The top surface 12c may be any material suitable for providing an impermeable interface on which the user may apply pressure to the second layer 12. The bottom layer 12a may be any material suitable for providing an interface between the first layer 11 and the central portion 12b of the second layer 12 such that the central portion 12b does not adhere to the first layer 11, the wound or the surrounding skin, which could be an issue upon detachment of the second layer 12 from the first layer 11, discussed below. It is envisioned that this second layer 12 may alternatively be composed of any number of layers and may include any materials suitable for these purposes described.

The first layer 11 is releasably attached to the second layer 12 such that, upon application to a wound, the second layer 12 can be detached leaving the first biodegradable layer 11 within and/or over the wound. The first and second layers can be attached by any process as desired. For example, the friction between the first and second layers may be sufficient to maintain them together until it is desired that the second layer 12 be detached. Alternatively, in a preferred embodiment, the first and second layers may be releasably attached via a plurality of weak adhesive connections, employing chemical or physical bonds, which may easily be detached by peeling the second layer 12 away from the first layer 11. While a peeling action is a preferred maneuver for detaching the second layer 12 from the first layer 11, other such maneuvers may also be used such as sliding, melting or otherwise adjusting the temperature, or the like. In one alternative, a weak adhesive, such as a biocompatible glue or the like, may be used to secure the two layers together. Such an adhesive may form a layer in between the first and second layer; the adhesive may be “spotted” onto the layers such that adhesion is only at specific points within the interface of the two layers, or the like. Any adhesive, whether synthetic or nonsynthetic may be used as desired. For example, a sugar or polysaccharide may be used to attach the first and second layers together, forming a weak, easily breakable bond between the two layers. The use of sugars may provide the added benefit that, upon wetting the first layer 11 and, optionally, the second layer 12, the sugar adhesive may dissolve such that the first and second layers are automatically detached from one another whereby the need for a peeling or like maneuver is minimized, which also minimizes any forces exerted on the first layer 11 which could separate the first layer 11 from the wound. It should be understood that the bandages of the present invention are provided in a variety of shapes and sizes to accommodate the various sizes and shapes of wounds. Exemplary shapes may be rectangular, circular, triangular, irregular, or the like. Additionally, as in the embodiments of FIGS. 2h, 2i, and 2j for example, the lattice structure 11h, 11i may also be divided into sub-units, such as layers, tiles, or both, and/or vary in height along its length or width dimension (as in FIG. 2h specifically).

Alternatively, such as in the embodiment of FIG. 2h, the interspersed lattice structure sheets 11h are each releasably attached to one another such that, upon application to a wound, the sheets 11h in contact with the wound can be detached from the sheets 11h not in contact with the wound, thereby leaving the wound-contacting sheets 11h within and/or over the wound. Each lattice structure sheet 11h may be attached to adjacent sheets 11h by any of the adhesive means disclosed in the paragraph above

Similarly, in the embodiment of FIGS. 2i and 2j, the lattice structure tiles 11i are each releasably attached along at least one side to adjacent tiles such that, upon application to a wound, the tiles 11i in contact with the wound can be detached from the tiles 11i not in contact with the wound, thereby leaving the wound-contacting tiles 11i within and/or over the wound. Each lattice structure tile 11i may be attached to adjacent tiles 11i by any of the adhesive means disclosed above. For example, in one embodiment, each tile may slightly overlap, along at least one side or edge, adjacent tile or tiles, and thus adhere to one another along such overlap. In another example, each tile may be adhered to secured to adjacent tiles along a perforated edge, or like structure, such that the tiles are releasably secured to one another.

The lattice structure of the first layer 11 may be formed by a variety of methods of manufacture. For example, in one embodiment, a lattice structure having a homogenous mixture of collagen and a clotting enzyme may be manufactured by drying a solution of collagen and a clotting enzyme, which, once dried, forms the solid, dry, porous, fluid-permeable, and biodegradable lattice structure. In one example, the solution of collagen and clotting enzyme may include thrombin as the clotting enzyme. One such solution that may be used in the present invention is Stryker Orthobiologics' Vitagel® Surgical Hemostat (manufactured by Orthovita, Inc., Malvern, Pa.). Vitagel® Surgical Hemostat or variations thereof are hemostats which include thrombin and collagen, as well as additional additives such as stabilizers or buffers and/or calcium and sodium. Vitagel® Surgical Hemostat or variations thereof may have properties suitable to both control bleeding and facilitate healing, while also interacting with the exudate to assist in clotting to thereby accelerate the healing and regeneration of the tissues of the wound. Other forms of Vitagel®, such as Vitagel® RT, may also be used in the present invention. Other suitable clotting enzymes may be used in the present invention such as those disclosed in U.S. Pat. Nos. 5,290,552; 6,096,309; 6,110,484; 6,277,394; 5,752,974; 5,616,689; 5,024,841; 5,997,811; and 5,110,604, each of which is hereby incorporated by reference herein as if fully set forth herein. It should be understood that, while Vitagel® Surgical Hemostat will be discussed as the preferred collagen-clotting enzyme solution, other biocompatible products may also be used within the scope of the present invention. In addition, it should be understood that various types of collagen may be used in the present invention, including for example native collagen or native-type collagen, pepsinized microfibrillar collagen (i.e., soluble collagen having minimal cross-linking), alkaline-modified (limed) collagen, and other forms such as the types of collagen made by the processes disclosed in U.S. Pat. Nos. 6,096,309, and 6,280,727, both of which are incorporated by reference herein as if fully set forth herein.

Continuing with this exemplary embodiment, the Vitagel® Surgical Hemostat may be placed in an appropriate container and dried. The drying step can be performed through a simple air drying process, through a vacuum-assisted dehydration process, or, in a preferred embodiment, through lyophilization (i.e., freeze drying). Other such drying processes may also be used. Upon drying, the Vitagel® Surgical Hemostat forms a solid, dry, porous, fluid-permeable, and biodegradable lattice structure suitable to be the first layer 11 of the bandage 10, such as is illustrated in FIGS. 2a and 2e.

In alternative embodiments of the present invention, as illustrated in FIGS. 2b and 2c, the first layer 11b, 11c of the multi-layered bandage 10b, 10c can be manufactured by combining a pre-formed lattice structure 111b with a solution containing at least a clotting enzyme 111c. For example, the first layer 11b, 11c may be manufactured by combining a pre-formed collagen lattice structure 111b with the above Vitagel® Surgical Hemostat solution. It is understood that a solution containing only a clotting enzyme may be used in these embodiments where a pre-formed lattice structure 111b, which already includes collagen, is used. This manufacturing method can result in a first layer 11b, 11c which is either homogenous throughout the lattice structure 111b (FIG. 2b), as described above, or heterogenous, such as a gradient-like structure throughout the lattice structure 111b (FIG. 2c).

In the first alternative as illustrated in FIG. 2b, a relatively larger volume of a clotting enzyme solution 111c is applied to the pre-formed collagen lattice structure 111b to thereby completely soak the lattice structure 111b and, therefore, form a homogenous collagen-clotting enzyme lattice structure. Once soaking is complete, the lattice structure lllb may be dried, as discussed above, to form the homogeneous, solid, dry, porous, fluid-permeable, and biodegradable collagen-clotting enzyme lattice structure 11b.

In the second alternative as illustrated in FIG. 2c, a relatively smaller volume of a clotting enzyme solution 111c is applied to the pre-formed collagen lattice structure 111b to thereby soak only a portion of the depth of the pre-formed lattice structure 111b, resulting in a gradiated or heterogenous lattice structure. It should be understood that the clotting enzyme solution 111c in the various embodiments may be thrombin or thrombin within a carrier, such as a collagen carrier.

As illustrated in FIG. 2d, the clotting enzyme solution 111c may be applied through a “drip” or “spot” process, as illustrated, or, alternatively, a soaking process in which the pre-formed lattice structure 111b is submerged in a volume of the solution 111c (though, particularly for the embodiment of FIG. 2c, the “drip” or “spot” process is used). Applying a certain volume of solution 111c to a targeted area of the lattice structure 111b will result in the “gradiated or heterogenous lattice structure” shown in FIG. 2c whereas applying a distributed volume of clotting enzyme (or using a soaking or submerging process) will result in the “homogenous collagen-clotting enzyme lattice structure” shown in FIG. 2b. Other such processes are also envisioned, such as infusion of the clotting enzyme solution 111c. It should be noted that, while FIG. 2d illustrates the application of the solution 111c while the second layer 12 is present and secured to the first layer 11b, 11c, this application process may be performed prior to positioning the first and second layers together.

FIG. 2e illustrates another alternative embodiment including a first layer 11e with a gradiated lattice structure. This gradiated lattice structure may be preformed collagen (as used in the embodiments of FIGS. 2b and 2c) to which the clotting enzyme solution 111c may be applied. Alternatively, this gradiated lattice structure may be formed from a similar process as used in the embodiment illustrated in FIG. 2a to form first layer 11a—specifically, by drying a solution of collagen and a clotting enzyme, which, once dried, forms the solid, dry, porous, fluid-permeable, and biodegradable lattice structure 111a. In this variation, however, the lattice structure 11e, while homogenous in terms of the distribution of collagen and clotting enzyme throughout the first layer 11e, may have a dense, closed portion at the wound-contacting surface and gradually becoming increasingly more porous toward the second layer 12.

The “heterogenous” lattice structure embodiments 10c, 10e may be more economical to commercialize because, for example, less clotting enzyme, and potentially collagen, is used. Further, the clotting enzyme that is present, as in embodiment 10c for example, is used in a more efficient manner because it is concentrated close to the wound-engaging portion of the first layer 11c. As such, the resulting first layer 11c, 11e of the bandage 10c, 10e may have a practical use for smaller wounds in which bleeding is easier to control. To the contrary, the above embodiments which form the “homogenous” lattice structure for a first layer of a bandage 11, 11a, 11b may be better suited for larger wounds, or wounds and/or patients prone to substantial bleeding.

In another embodiment of the present invention, as illustrated in FIGS. 2f and 2g, the first layer 11f of the multi-layer bandage 10f includes a plurality of channels 13 which pass through at least a portion of the first layer 11f and preferably may extend through the entire height of the first layer 11f between the top surface and the bottom surface. The channels 13 are formed in the lattice structure, and thus are in addition to the natural porosity of the lattice structure. The channels 13 may be substantially linear, as illustrated, though they may also include curves or bends along their length as desired. The channels 13 can provide improved flow characteristics of the exudate into the lattice structure, which may be particularly beneficial in bandages which have a homogenous first layer such that the clotting enzyme, throughout the first layer, can be more efficiently utilized.

Continuing with the embodiment illustrated in FIGS. 2f and 2g, positioned at the top surface of the first layer 11f, may be a plurality of flaps 14 which are hingedly connected to the top surface to alternatively cover the adjacent openings of the channels 13 or maintain an open end of the channels 13 at the top surface. The flaps 14 may regulate the amount of fluid interaction between the first layer 11f and the second layer 12 including, namely, the amount of exudate flowing from the first layer 11f to the second layer 12. In doing so, the flaps 14 serve to maintain an amount of exudate within the first layer 11f to optimally facilitate hemostasis and overall wound healing and to allow only excess exudate to be absorbed into the second layer 12.

In still another alternative embodiment as illustrated in FIG. 2h, multi-layered bandage 10h includes a plurality of lattice structure sheets 11h. Generally, this embodiment differs from others disclosed herein as a plurality of lattice structure sheets 11h are stacked atop one another while a second layer (similar to the second layer 12 of FIG. 1) is optionally not present. As explained above, each sheet 11h is also releasably attached to adjacent sheets 11h, such that as exudate flows through the bandage 10h, certain of the sheets 11h in contact with the exudate can facilitate hemostasis, while certain of the other sheets 11h not in contact with exudate can be subsequently detached without disturbing the healing process.

In a similar embodiment illustrated in FIGS. 2i and 2j, multi-layered bandage 10i includes a plurality of lattice structure tiles 11i. As shown, a plurality of lattice structure tiles 11i are arranged side-by-side and, optionally, also stacked atop one another, while a second layer (similar to the second layer 12 of FIG. 1) is secured above the tiles 11i (second layer not present for sake of clarity). As explained above, each tile 11i is also releasably attached to one or more adjacent tiles 11i, such that as exudate flows through the bandage 10i, certain of the tiles 11i in contact with the exudate can facilitate hemostasis, while certain of the other tiles 11i not in contact with exudate can be subsequently detached without disturbing the healing process. In a preferred embodiment, the tiles over the wound would remain in place while those tiles outside of the wound area, assuming no overflow of exudate, would be removed, thus a bandage 10i of a similar surface area to the wound would remain. Along these lines, it is envisioned that the tiles may have a shape other than rectangular, as illustrated, and may also be a random assortment of shapes such that the resulting tiles 11i, left in the wound, can more closely mimic the typically random shape of a wound.

For the embodiments of FIGS. 2h, 2i, and 2j, the lattice structure within each sheet 11h or tile 11i may be formed from any of the above-discussed processes, such as from a dried homogeneous collagen-clotting enzyme solution 111a as in FIGS. 2a and 2e, or a collagen lattice structure 111b with a clotting enzyme solution 111c applied throughout as in FIG. 2b. As such, each lattice structure sheet 11h or tile 11i is solid, dry, porous, fluid-permeable, and biodegradable.

Furthermore, the structures of the embodiment of FIGS. 2h allows for the entire thickness of the bandages 10h to be composed of a porous, fluid-permeable, and biodegradable lattice structure, thereby suiting the embodiments particularly well for applications in which a wound produces relatively large amounts of exudate or where the amount of expected exudate is unknown. Both situations are frequently encountered in, for example, emergency medical procedures, deep tissue wounds, internal wounds, or the like.

Additionally, in any of the above embodiments, additional characteristics may also be included. For example, the first layer 11 of the bandage may include a skin-colored pigment such that, subsequent to the removal of the second layer 12, the first layer 11, which is left on the wound, can be made to resemble the pigment of the surrounding skin. Any skin-tone pigment may be included as is known in the art. Preferably, such pigment is positioned only on the top surface of the first layer 11, though it may also be positioned throughout as desired.

Moreover, the first layer 11 of the bandage may also include a foaming agent, bubbling agent, or the like, or in any combination. These various agents may provide assistance in mixing the exudate with the clotting enzyme in the first layer. For example, upon application of the first layer 11 to the wound, and upon the exudate infiltrating the first layer 11, the foaming or bubbling agent may chemically react with the exudate to form foam and/or bubbling to assist in mixing the clotting enzyme with the exudate.

The present invention also includes embodiments of various methods of use of the multi-layered bandage of the various above embodiments. While the below discussion of the various methods are discussed using the embodiment of bandage 10, any of the various described embodiments of a bandage having first and second layers may be used in any of the methods.

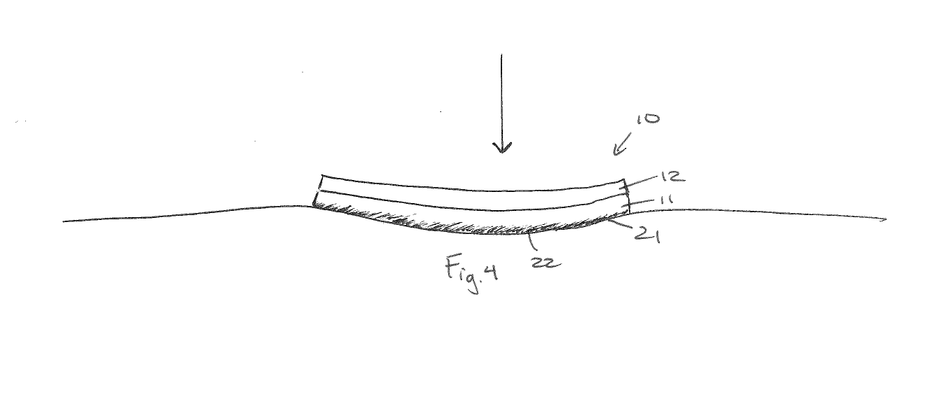

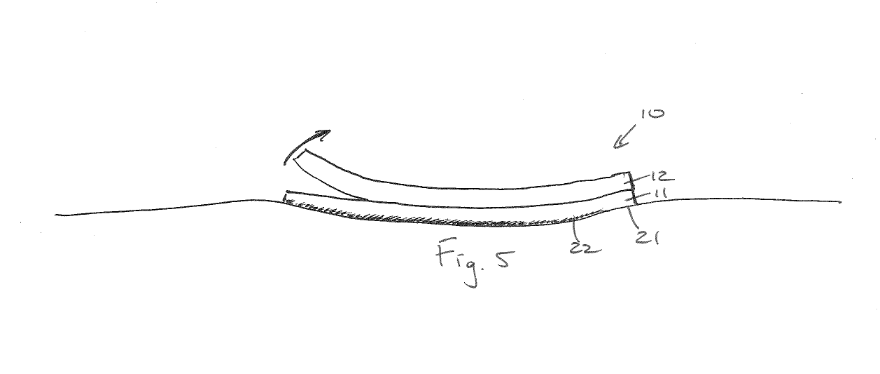

Illustrated in FIGS. 3-5 is one embodiment of a method of use of the multi-layered bandage 10. In this embodiment, the bandage 10 is administered to a patient in need thereof with the first layer 11 in contact with a wound and wound exudate 22 (FIG. 3), whereupon a compressive force is placed against the second layer 12 toward the wound (FIG. 4). This compressive force translates to the first layer 11, thereby causing the exudate 22 to flow into the first layer 11 (as in FIG. 4) and, optionally, therethrough to be absorbed into the second layer 12. Contact between the exudate 22 and the clotting enzyme of the first layer 11 initiates hemostasis.

In accordance with the present invention, the collagen and clotting enzyme lattice mixture of the first layer 11 may promote hemostasis and wound healing and can remain in place on the wound 21 throughout the healing process. The porous structure of the first layer 11 also may act as a reservoir for exudate 22, thereby retaining the exudate 22 in contact with the collagen-clotting enzyme lattice. Further, the first layer 11 may serve to retain growth factors in contact with the wound 21 which may further expedite clotting and promote healing. As healing progresses, the first layer 11 may continuously biodegrade and be absorbed and replaced by new tissue. One of the primary functions of the present invention bandage 10 is to interact with the wound to form biologic bridging between the wound 21 and the lattice structure in order to expedite clotting, achieve hemostasis, and remove excess exudate 22 from the site. This is achieved via the hemostatic and enzymatic components of the biodegradable first layer 11 and the non-resorbable second layer 12. In use and upon application of the bandage 10, the materials of the first layer 11 engage the wound 21 to form a clot and achieve hemostasis. Excess blood 22 exudes through the first layer 11 into the second layer 12, which is removed from the first layer 11 shortly upon application. The first layer 11 is left at the site to further heal the wound 21. Resorption of the first layer 11 is concurrent with healing.

Upon applying compression, as in FIG. 4, excess exudate 22 may flow to the second layer 12, whereupon the second layer 12 serves both as a sponge for the exudate 22 and a barrier to infection and foreign contaminants. In addition, in one alternative, the second layer 12 may also serve as a reservoir for liquids, positioned in the second layer 12 prior to application to a patient in need thereof, to hydrate the wound 21.

As illustrated in FIG. 5, and as described above, the second layer 12 is releasably attached to the first layer 11. As hemostasis occurs throughout the first layer 11, the first layer 11 is continuously resorbed into the wound 21. At a time deemed to be safe, the second layer 12 may be detached from the first layer 11 and discarded. In a preferred embodiment, the second layer 12 is removed from the first layer 11 shortly after application to the wound 21, such that cell adhesion to the second layer 12 is minimized if not altogether prevented, and thus allows for simplified detachment from the first layer 11 without disturbing the healing process.

In one example, the second layer 12 is removed from the first layer 11 shortly after hemostasis is achieved. When the bandage 10 of the present invention is used, hemostasis may occur at the interface of the first layer 11 with the wound 21, e.g., where the exudate 22 contacts the clotting enzyme on the bottom surface of the first layer 11. Hemostasis may also occur within the lattice structure, as exudate 22 migrates into the lattice structure of the first layer 11, though such hemostatic activity may be confined to the portion of lattice structure directly adjacent to the bottom surface of the first layer 11. However, if a bandage 10 having a homogeneous lattice structure, as described above, is used, hemostasis can occur anywhere within the first layer 11 as clotting enzyme is present throughout the volume of the lattice structure.

Typically, hemostasis is achieved within minutes, though the particular time required is dependent on the size and location of the wound 21 (e.g., the level of vascularization in the wound area), as well as specific characteristics of the patient (e.g., clotting ability, blood pressure, etc.). Thus, for relatively smaller, shallower, and/or drier wounds, it is preferred that the second layer 12 be removed from the first layer 11 within 30 minutes of application to the wound 21, preferably within 15 minutes of application to the wound 21, more preferably within 5 minutes of application to the wound 21 and even more preferably within 2 minutes of application to the wound 21. However, for larger and/or deeper wounds or wounds with heavy blood flow, the second layer 12 may remain in place for at least 30 minutes, preferably at least 15 minutes, though as before, once hemostasis is achieved, the second layer 12 may be removed.

In an alternate embodiment of a method of use, the multi-layered bandage 10 is administered to a patient in need thereof with the first layer 11 in contact with an internal wound (not shown). Such an internal wound may include an injury or other damage to an organ or other internal tissues. Other damages on internal or superficial tissues resulting in deep wounds including ulcers, burns, stasis dermatitis, or the like. Upon administration of the multi-layered bandage 10, exudate is absorbed by the first layer 11, and optionally, the second layer 12 while compression is optionally applied to the second layer 12, which is translated into the first layer 11. Upon achieving hemostasis, the second layer 12 may be immediately detached and discarded and the wound may be sutured or otherwise closed, and/or bandaged with additional material if required.

In such embodiments where the bandage 10 is used in an internal application, or in a deep skin wound, the first layer 11 can be considered to be a biodegradable implant including a homogenous mixture of collagen and a clotting enzyme forming a porous lattice structure. Since it is typical that there will be excessive blood flow in such wounds or injuries, it is preferred that the implant include the clotting enzyme throughout the lattice structure in a homogenous manner (such as in FIGS. 1, 2a, and 2b) as added clotting ability will likely be required.

This embodiment also includes an applicator releasably secured to the implant, the applicator including an absorptive matrix and an interface adapted to allow a user to apply compression through the applicator and against the implant.

In yet another embodiment, the present invention includes a method of treating a wound 21 using the multi-layered bandages 10h, 10i of FIGS. 2h, 2i and 2j. Upon applying either bandage 10h, 10i to the wound 21, compression is applied through the bandage 10h, 10i and to the wound 21. Exudate 22 may then flow and migrate from the wound 21 to and into the bandage 10h, 10i such that the exudate 22 contacts at least one of the sheets 11h or tiles 11i. Compression is maintained until hemostasis is achieved. Hemostasis may be achieved at or within the at least one sheet 11h or tile 11i which contacts the exudate 22. Once hemostasis is achieved, compression is removed and the sheets 11h or tiles 11i which did not contact the exudate 22 are removed.

Continuing with the embodiment with specific reference to the embodiment of FIG. 2h, the removed sheets 11h may then be discarded, or, if the patient has multiple wounds 21, the formerly removed sheets 11h may then be applied to a second wound 21, and the above steps of the method can be repeated. In some examples, such a method may be useful for wounds of unknown exudate capacity, in emergent care situations where a patient has multiple wounds, or during a surgical procedure in which the bandage 10h can be used internally.

Continuing with the embodiment with specific reference to the embodiment of FIGS. 2i and 2j, the removed tiles 11i may then be discarded (along with the second layer of the bandage, as discussed above—second layer not shown for purposes of clarity), and the tiles 11i left in the wound can substantially mimic the shape of the wound 21, though of course, if a larger amount of exudate flowed from the wound, the tiles 11i left at the wound may extend to an area outside of the area of the wound. Also, while a single-layer height of tiles 11i is preferred, if multiple layers of tiles are present (as in FIG. 2i), certain of the layers can also be removed as discussed above with reference to FIG. 2h.

Other similar methods of use of the present invention are also envisioned to assist in controlling and repairing a wound in a patient in need thereof.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A multilayer bandage comprising
a first layer for contacting a wound comprising a porous, fluid-permeable, biodegradable lattice structure formed from a homogeneous mixture of collagen and a clotting enzyme; and
a second layer releasably secured to the first layer comprising an absorbent material,
wherein the bandage is adapted to allow flow of a fluid from the wound into the first layer to interact with the clotting enzyme to promote clot formation and, optionally, through the first layer and into the second layer.

2. The bandage of claim 1, wherein the clotting enzyme includes thrombin.

3. The bandage of claim 1, wherein the collagen is native or microfibrillar.

4. The bandage of claim 1, wherein the second layer is non-resorbable.

5. The bandage of claim 1, wherein at least one of the first and second layers includes a skin-tone pigment.

6. The bandage of claim 1, wherein the second layer includes an absorptive matrix and an interface adapted to allow a user to apply compression through the second layer and against the first layer.

7. The system of claim 1, wherein the first layer further includes polyethylene glycol.

8. The system of claim 1, wherein the porous lattice of the first layer allows for integration between the clotting enzyme and exudate from the wound.

9. The system of claim 1, wherein the first layer further includes a mixing agent.

10. The system of claim 9, wherein the mixing agent includes a foaming agent, a bubbling agent, or both.

11. The system of claim 1, wherein the first layer further includes a plurality of channels extending substantially linearly through a thickness of the first layer between open ends at a bottom surface of the first layer, adjacent the wound, and open ends at a top surface of the first layer, opposite the bottom surface.

12. The system of claim 11, wherein at least one of the channels includes a flap, secured to the top surface, and, in a closed position, is adapted to cover at least one of the open ends of the channels at the top surface.

13. A method for treating a wound comprising the steps of
applying a bandage having a first layer and a second layer,
the first layer including a porous, biodegradable lattice structure and a clotting enzyme for contacting the wound, the second layer including an absorbent matrix, the second layer is releasably secured to the first layer;
applying compression to a top surface of the second layer such that a compressive force transfers through the absorptive matrix, through the lattice structure and against the wound, thereby allowing exudate from the wound to migrate into the lattice structure and, optionally, into the absorptive matrix;
maintaining the compressive force until hemostasis is achieved, and
once hemostasis is achieved, releasing the second layer from the first layer, thereby leaving the first layer in contact with the wound.

14. The method of claim 13, wherein a portion of the exudate migrates through the first layer and is absorbed by the second layer.

15. The method of claim 13, wherein the hemostat includes thrombin.

16. The method of claim 13, wherein the compressive force is applied for less than about 5 minutes.

17. The method of claim 16, wherein the compressive force is applied between about 1 minute and about 3 minutes.

18. The method of claim 17, wherein the step of releasing the second layer occurs immediately after hemostasis is achieved.

19. The method of claim 13, wherein, during compression, hemostasis occurs at an interface between the first layer and the wound, within the lattice structure, or both.

20. A multi-layer bandage comprising
a first layer for contacting a wound comprising a dry, porous, fluid-permeable, and biodegradable lattice structure, wherein at least a portion of the lattice structure is infused with a clotting enzyme; and
a second layer releasably secured to the first layer comprising an absorbent material;
wherein the bandage is adapted to allow flow of a fluid from the wound into the first layer to interact with the clotting enzyme to promote clot formation and, optionally, through the first layer and into the second layer.

21. The bandage of claim 20, wherein the portion of the lattice structure infused with the clotting enzyme is positioned adjacent the wound.
